(19) United States
(12) Patent Application Publication
(10) Pub. No.: US 2005/0175685 A1
(43) Pub. Date: Aug. 11, 2005

(54) Universal pet medication and capsule system

(76) Inventor: Jeff Rock Grandinetti, Edmonton (CA)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/777,273

(22) Filed: Feb. 11, 2004

(51) Int. Cl. A61K 9/48

(52) U.S. Cl. 424/451

(57) ABSTRACT

A pet medication capsule is of uniform cylindrical form, closed at one end and open at an opposing end. The capsule is made of an edible product attractive to an animal and has a length of between 7/16 and ⅞ inches inclusive and a diameter of approximately ½ inches. A round interior wall surface has a diameter of approximately ¼ inches and a length of between ⅜ and ¾ inches. A solid medication has a girth of such size as to be secured within the capsule by wedging action against the interior wall surface of the capsule.

### BACKGROUND OF THE INVENTION

### INCORPORATION BY REFERENCE

Applicant(s) hereby incorporate herein by reference, any and all U. S. patents and U.S. patent applications cited or referred to in this application.

1. Field of the Invention

This invention relates generally to pet medication delivery systems and particularly to an edible capsule and medication system that uses size and shape to achieve objectives.

2. Description of Related Art

It is well known that pet such as cats and dogs are adverse to swallowing medications. If the medication is of such taste as to be rejected, the pet will refuse to approach. Even if the medication is tasteless, it is found that pets will often refuse to accept small medicaments when offered without a delivery system. Frequently, the medication is place into the pet's food dish as feeding time and swallowed with food in the animals haste to ingest its dinner. When this is not possible, due to a wary animal, or where medication is require several times daily or not at normal feeding times, it is necessary to further trick the pet in order to deliver medication. A related problem is that when a medication carrier is too large, the pet will attempt to chew it and may thus find the medication and reject it, or if even if the medication is not rejected, upon chewing, the carrier may open, the medication may drop out and fall to the floor without being swallowed. The prior art has attempted to deal with this area of difficulty as described in the following:

Mamajek et al., U.S. Pat. No. 4,207,890 describes a drug-dispensing device and method for controlling and prolonging internal administration of medicaments to warm-blooded animals comprising an outer polymer envelope containing an expanding agent, drug metering means, and the drug itself. The outer polymer envelope is permeable to both the drug and body fluids and expands when the expanding agent is contacted by body fluids when the device is in the environment of use (e.g., the stomach). This expansion maintains the device in the environment of use while the drug is administered by the metering means.

Harold, U.S. Pat. No. 4,857,333 describes an animal food product for administering medication to animals that is a formed, chewable food treat with at least one pre-formed pocket therein opening to an outer surface of the treat and sized to retain a medicant therein.

Boyer, U.S. Pat. No. 5,100,651 describes a health product, in particular for the caring of the teeth of a dog, constituted by a support incorporating or having absorbed various watersoluble active elements or constituents, this support being made from a natural or synthetic digestible material suitable for being gnawed, chewed or licked by the dog.

Simone et al., U.S. Pat. No. 5,296,209 describes an edible pet chew product having a flexible cellular matrix in which is contained a cellulosic fibrous material such as corn cob fractions having a mechanical cleansing function, which when chewed by the pet, effects a reduction in plaque, stain and tartar on the pet's teeth. An oral care additive may be incorporated in the matrix to inhibit dental problems, the composition of the cellular matrix being substantially inert to the oral care additive.

Wesenhagen, U.S. Pat. No. 5,674,515 describes a food item and method for administering medication to a subject, particularly a dog wherein the food item is a short edible tube with an interior chamber having a closed end and open end providing that the medication may be inserted into the chamber through the open end of the tube and then the open end of the tube is pinched closed. The food item is composed of foodstuffs such as meats, a congealing agent, if necessary, such as flour and water, with a percentage of water selected to maintain a consistency of the foodstuff that maintains the shape of the tube in a handling operation yet is readily deformed by pinching the open end.

Baumgardner, Sr., U.S. Pat. No. 5,792,470 describes a new Edible Container For Administering Medication To Animals for administering medication to a pet without the pet realizing the presence of the medication, where the medication may comprise pills, powder, liquid, or other substances. The inventive device includes a length of a swaged tubular member the size of which the animal may consume and where the swaged tubular member is constructed from an tasty edible material which conceals the medication within, and an impermeable wrapper removably encloses the length of swaged tubular member.

Durand et al., U.S. Pat. No. 5,853,757 describes a carrier for pills, tablets, or capsules allowing for ease of oral consumption by animals. The carrier having a preformed chamber allowing for the insertion of medication therein, the carrier is deformable causes securement of the medication within the chamber. The carrier masks the scent of medication and including a lubricant to allow for ease of consumption of the carrier and contained medication.

Meyer, U.S. Pat. No. 5,895,662 describes a hollowed pet treat for medicating a pet including a vial constructed entirely of an edible product and defining an interior space and an open top. Next provided is a cap formed entirely of the edible product for being removably coupled to the open top of the vial for containing a predetermined medicine within the interior space.

Axelrod, U.S. Pat. No. 6,110,521 describes a completely digestible highly nutritious dog chew formulated primarily of wheat flour, casein and starch carbohydrate, the texture of hardness of which is easily modified to suit a particular dog by the dog owner. By irradiating the chew in a microwave oven, the chew is caused to expand and is thereby rendered more easily chewable.

Hayden et al., U.S. Pat. No. 6,143,316 describes a digestible pouch and method for aiding in the oral administration of substances suitable for ingestion, in particular medicinal agents. Specific embodiments of the invention include a pouch formed of an edible food stuff material that is pleasing to the recipient, wherein the pouch comprises an inner compartment area for housing the particular substance prior to oral administration. The inventive pouch is particularly useful in administering medications to common domestic animals and children to whom oral administration of such medications is often difficult.

Kelly et al., U.S. Pat. No. 6,265,011 describes a dog biscuit for controlling malodorous breath in dogs. The dog biscuit includes about 8-12 parts by weight whole wheat flour, about 4-6 parts oat bran, about 2-3 parts non-fat dry milk, and about 2-3 parts brown rice flour. The dog biscuit also includes water, vanilla extract, parsley, alfalfa powder, vegetable oil, peppermint extract, tarragon, peppermint leaf powder, and spearmint leaf powder. The vegetable oil includes an antioxidant and is preferably sunflower oil. The parsley is preferably fresh parsley, and the ratio of parsley to whole wheat flour is about 1:4 by weight. The dog biscuit is readily eaten by dogs and effectively neutralizes malodorous breath in them. Also part of the invention is a method of making the dog biscuit.

Wang, U.S. Pat. No. 6,455,083 describes an edible thermoplastic made from about 30 to 50 wt. % protein comprising a mixture of plant and animal derived protein, about 20 to 50 wt. % starch about 10 to 20 wt. % water, about 1 to 10 wt. % edible fiber, and about 0.5 to 3 wt. % metallic salt hydrate. When molded, the thermoplastic has good strength and stiffness and other physical properties. The edible thermoplastic may be molded in a variety of shapes including a segmented nutritional pet chew with a plurality of segments separated by a plurality of scores. The scores serve to structurally weaken the pet chew so that it may be broken into smaller pieces. When molded the edible thermoplastic has a density of about 1.2 to 1.5 g/cubic centimeters.

Our prior art search with abstracts described above teaches: a meat product pet medicine capsule, a digestible pouch and method for administering medications to an animal, an edible container for administering medication to animals, a carrier for animal medication, a food product for administering medication to animals, a pet chew product having oral care properties, a method and product for dispensing medication for animals, a health product in particular for the caring of the teeth of dogs, a wheat and casen dog chew with modifiable texture, a dog biscuit for controlling malodorous breath in dogs, an edible thermoplastic and nutritious pet chew, and a drug dispensing device and method, but does not teach a combination medication capsule and medication that are able to mount the medication by wedging action, that are of such size as to be swallowed without chewing and are able to trick a pet into taking the medication without knowing it. The present invention fulfills these needs and provides further related advantages as described in the following summary.

### SUMMARY OF THE INVENTION

The present invention teaches certain benefits in construction and use which give rise to the objectives described below.

A pet medication capsule is of uniform cylindrical form, closed at one end and open at an opposing end. The capsule is made of an edible product attractive to an animal and has a length of between 7/16 and ⅞ inches inclusive and a diameter of approximately ½ inches. It has been found that capsules in this size range are able to be swallowed whole by typical cats and small to medium sized dogs. This avoids the problems that arise when the capsule is chewed. A round interior wall surface has a diameter of approximately ¼ inches and a length of between ⅜ and ¾ inches. A solid medication has a girth of such size as to be secured within the capsule by wedging action against the interior wall surface of the capsule. The capsule is of a relatively soft edible foodstuff such that the medication, normally a pill or caplet, is able to be engaged within the capsule by wedging action so that no cap or cover is necessary.

A primary objective of the present invention is to provide an apparatus and method of use of such apparatus that yields advantages not taught by the prior art.

Another objective is to provide such an invention of such size and type as to trick a pet such as a cat or dog to swallow a medication without realizing it.

A further objective is to provide such an invention capable of securing a medication within a capsule without requiring a cap or cover.

A still further objective is to provide such an invention of such size as to be swallowed by a pet without chewing it.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective exploded view of the invention;

FIG. 2 is a perspective view thereof showing a combined capsule and medication;

FIG. 3 is a cross-sectional view thereof as taken along line 3-3 in FIG. 2; and

FIG. 4 is a perspective view thereof showing the manner in which the invention is introduced to a dog.

### DETAILED DESCRIPTION OF THE INVENTION

The above described drawing figures illustrate the invention in at least one of its preferred embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

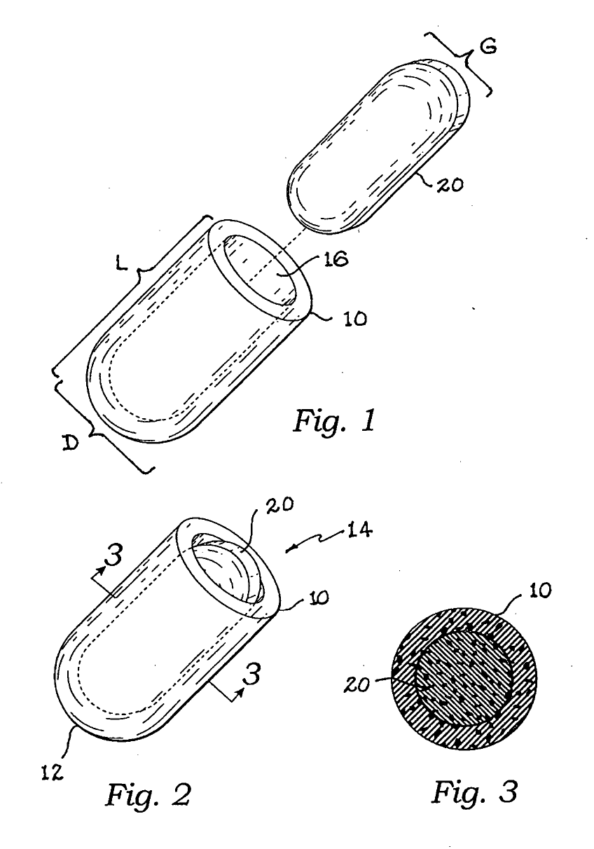

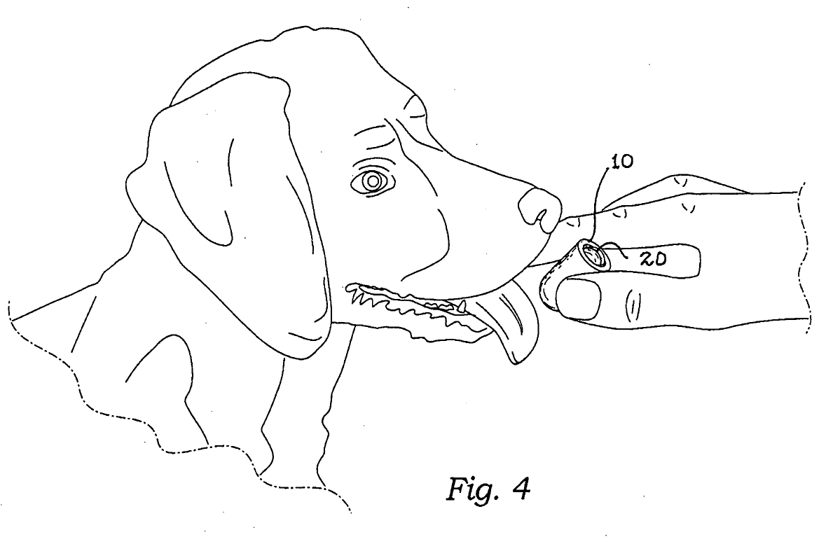

The present invention is a combination of a round capsule 10 and a solid medication 20 in pill or caplet form or the equivalent thereof, as normally used for pets and other animals. Such medications 20 shall mean those as used for hygiene, medication, diet supplement and any other reason, but shall not be taken to mean the normal diet foodstuffs that constitutes the animal's diet. Although, pets are most frequently mentioned herein, the present invention is highly applicable to farm, zoo and even wild animals of all kinds. The capsule 10 is preferably of a uniform cylindrical shape, closed at one end 12 and open at an opposing end 14. The capsule 10 is made of an edible product attractive to a pet or other animal, such as a baked cookie batter or dough. The capsule is between 7/16 and ⅞ inches in length “L”, inclusive, and has a diameter “D” of approximately ½ inches. These dimensions are considered critical to meeting the objectives of this invention and it has been found that use of the invention within the boundaries of such dimensions is able to meet the needs of most small to medium sized pets, a rather surprising result. A round interior wall surface 16 has a diameter slightly smaller than the girth of the medication it is to be used with, and a length equal to or slightly longer than the length of the medication. “Girth” as used herein shall mean the largest dimension of the medication 20 as measured laterally across the medication, see dimension “G” in FIG. 1. It is the extremities of this girth dimension “G,” that most firmly engage the interior wall surface 16 of the capsule 10.

The medication 20 is generally solid and hard or at least maintains a regular shape elastically. The medication has its girth dimension “G” of such size as to be secured within the capsule 10 by wedging action against the interior wall surface 16 of the capsule 10. Such wedging action may take one of two forms, as follows.

The medication 20 may be harder than the interior wall 16 of the capsule 10 so as to dislodge a portion of the interior wall 16 when the medication 20 is forced into the capsule 10, and is therefore held in place by friction.

The medication 20 may alternately be soft and resilient, such as a gel caplet, and may, in this instance, be secured within the capsule 10 by plastic strain developed upon deforming the medication 20 when it is forced into the capsule 10.

Through our research we have found that most cats and small to medium sized dogs will devour a capsule 10 of a size and type as described here without chewing it so as to eliminate the worry of rejection or loss of the medication 20. It has been found that it is beneficial to use the capsule 10 alone, i.e., without the medication so as to train the pet to accept the capsule 10 as a treat. Thus, when it is necessary to administer the medication 20, the animal will readily take the treat without consideration of the medication 20 hidden within.

The enablements described in detail above are considered novel over the prior art of record and are considered critical to the operation of the instant invention and to the achievement of the above described objectives. The words used in this specification to describe the invention and its various embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of this described invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments below or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. An apparatus comprising in combination: a medication and a round capsule of uniform cylindrical form, closed at one end thereof and open at an opposing end thereof; the capsule made of an edible product attractive to an animal; the capsule having a length of between 7/16 and ⅞ inches inclusive, a diameter of approximately ½ inches, and a round interior wall surface having a diameter slightly smaller than the girth of the medication and a length of at least equal to the length of the medication; the medication being in solid form and positioned within the wall surface of the capsule by wedging action.

2. The apparatus of claim 1 wherein the medication is harder than the interior wall of the capsule, and is of such size as to dislodge a portion of the interior wall when the medication is forced into the capsule.

3. The apparatus of claim 1 wherein the medication is soft and resilient and is of such size as to be secured within the capsule by plastic strain developed upon placing the medication within the capsule.

4. An apparatus comprising a round capsule of uniform cylindrical form, closed at one end thereof and open at an opposing end thereof; the capsule made of an edible product attractive to an animal; the capsule having a length of between 7/16 and ⅞ inches inclusive, a diameter of approximately ½ inches, and a round interior wall surface having a diameter of a size for engaging a medication by wedging action.
